(19) United States
(12) Patent Application Publication
(10) Pub. No.: US 2024/0009453 A1
(43) Pub. Date: Jan. 11, 2024

(54) CARDIAC PACING LEADS AND DELIVERY SYSTEM

(71) Applicant: Singular Medical (USA) Inc., Irvine, CA (US)

(72) Inventors: Yongxing Zhang, Irvine, CA (US); Martin Tze, Irvine, CA (US); Scott Hayden, Irvine, CA (US); Ryan Bauer, Irvine, CA (US); Entao Liu, Irvine, CA (US)

(21) Appl. No.: 17/811,004

(22) Filed: Jul. 6, 2022

(51) Int. Cl. A61N 1/05

(52) U.S. Cl. CPC A61N 1/0573

(57) ABSTRACT

A lead distal tip for a pacemaker includes a lead body including an insulated wire having a diameter of 2 French or less. The small size and flexibility of the lead distal tip and lead body reduce penetration and dislodgement risk for the lead distal tip. The lead distal tip can be delivered using a delivery catheter engaging with features on the lead distal tip to allow for the guidance and securement of the lead distal tip, despite its small and flexible nature. The attachment of the lead distal tip to heart tissue can be achieved by extending a linear member and/or a helical member from an end of the lead distal tip using features provided on the delivery catheter.

### FIELD

This disclosure is directed to leads for cardiac pacing, especially conduction system pacing, and delivery systems for such leads.

### BACKGROUND

An implantable pulse generator (e.g., an implantable pacemaker, an implantable cardioverter-defibrillator, etc.) is a medical device powered by a battery, contains electronic circuitry having a controller, and delivers and regulates electrical impulses to an organ or a system such as the heart, the nervous system, or the like. A lead is a thin, flexible, electrical wire connecting a device such as the implantable pulse generator to a target such as the organ or system, transmits electrical impulses (e.g., a burst of energy) from the device to the target, and/or senses or measures the potential or the voltage from the target. A catheter is a tubular medical device for insertion into canals, vessels, passageways, or body cavities usually to keep a passage open to facilitate the delivery of e.g., a lead or leads during a surgical procedure. The process of inserting a catheter is “catheterization”. The conduction system of the heart consists of cardiac muscle cells and conducting fibers that are specialized for initiating impulses and conducting the impulses through the heart. The cardiac conduction system initiates the normal cardiac cycle, coordinates the contractions of cardiac chambers, and provides the heart its automatic rhythmic beat. Conduction system pacing (CSP) is a technique of pacing that involves implantation of pacing leads along different sites or pathways of the cardiac conduction system and includes His-bundle pacing, left bundle branch pacing, right bundle branch pacing, and/or bilateral pacing (pacing both the left bundle branch and the right bundle branch).

In chronic implantation, CSP leads in the cardiac chamber and the lead electrodes in the interventricular septum (IVS) or interatrial septum (IAS) move with and relative to the myocardium.

### SUMMARY

This disclosure is directed to leads for cardiac pacing, especially conduction system pacing, and delivery systems for such leads.

By using a highly flexible and miniature diameter lead body with a miniature distal tip and electrode, the possible lead perforation and dislodgement issues for CSP leads resulting from cardiac dynamics can be mitigated, it may enhance the performance and longevity of the CSP systems. In particular, the flexibility and smaller size of the leads can reduce the force exerted by the lead during cardiac motion, thus improving stability and electrical performance of the lead. Further, using miniature, highly flexible leads reduces obstruction to veins and valves such as the tricuspid valve that are along the length of the lead. The reduced effects on the tricuspid valve can reduce regurgitation or the formation of fibrotic tissue or calcification at or near the valve, and thus preserve valve functionality.

A delivery system can provide the flexible lead body with the pushability, torquabiliy, and steerability required to successfully introduce the CSP lead into the cardiac tissue, allowing the lead to possess the increased flexibility and smaller size without compromising the ability to successfully place the leads in cardiac tissue.

In an embodiment, a lead distal tip for a pacing system includes a lead distal tip body having a first end and a second end, opposite the first end. The lead distal tip further includes a linear member disposed at the first end and a helical member surrounding the linear member. The lead distal tip also includes a ring electrode disposed on the lead distal tip body and a lead body extending from the second end, wherein the lead body has an outer diameter of 2 French or less.

In an embodiment, the lead distal tip further includes an engagement feature configured to engage with a delivery tool. In an embodiment, the engagement feature includes a socket formed at the second end. In an embodiment, the socket is configured to receive a stylet of the delivery tool. In an embodiment, the engagement feature includes a plurality of depressions in an outer surface of the lead distal tip body. In an embodiment, the engagement feature includes a plurality of projections disposed on an outer surface of the lead distal tip body.

In an embodiment, the linear member is extendable and retractable. In an embodiment, the helical member is extendable and retractable.

In an embodiment, the lead distal tip further includes a second ring electrode disposed on the lead distal tip body.

In an embodiment, a pacemaker lead and delivery system includes a lead distal tip including a lead distal tip body having a first end and a second end, opposite the first end, a linear member disposed at the first end, a helical member surrounding the linear member, a ring electrode disposed on the lead distal tip body, a lead body extending from the second end, wherein the lead body has an outer diameter of 2 French or less, and a tip engagement feature. The system further includes a lead delivery catheter including a rigid body segment and a delivery catheter engagement feature configured to engage the tip engagement feature.

In an embodiment, the tip engagement feature includes a socket and the delivery catheter engagement feature includes a stylet. In an embodiment, at least one of the linear member and the helical member are extendable and retractable, and the stylet is configured to extend or retract said at least one of the linear member and the helical member.

In an embodiment, the delivery catheter engagement feature includes recesses in an inner surface of a distal end of the lead delivery catheter, and the tip engagement feature includes projections from an outer surface of the lead distal tip body.

In an embodiment, the delivery catheter engagement feature includes projections from an inner surface of the lead delivery catheter, and the tip engagement feature includes recesses formed in an outer surface of the lead distal tip body. In an embodiment, a distal end of the delivery catheter is configured to be deflected such that the delivery catheter engagement features interface with the tip engagement features. In an embodiment, the system further includes grasping lines extending from the distal end of the delivery catheter, the grasping lines configured to deflect the distal end of the delivery catheter when tension is applied to the grasping lines.

In an embodiment, a method of implanting a pacemaker lead in a subject includes engaging tip engagement features of a lead distal tip with delivery catheter engagement features of a delivery catheter, directing the lead distal tip to a target location while the tip engagement features and the delivery catheter engagement features are engaged, attaching the lead distal tip to heart tissue of the subject at the target location, and disengaging the tip engagement features from the delivery catheter engagement features after the lead distal tip is attached to the heart tissue. The lead distal tip further includes a lead distal tip body having a first end and a second end, opposite the first end, a linear member disposed at the first end, and a helical member surrounding the linear member. The lead distal tip also further includes a ring electrode disposed on the lead distal tip body and a lead body extending from the second end, where the lead body has an outer diameter of 2 French or less.

In an embodiment, the target location is interventricular septum (IVS) or interatrial septum (IAS) of the subject.

In an embodiment, attaching the lead distal tip to the heart tissue of the subject includes extending one or more of the linear member and the helical member into the heart tissue.

### DRAWINGS

FIG. 1 shows a lead according an embodiment.

FIG. 2 shows a lead according to another embodiment.

FIG. 3 shows a lead and a delivery system according to an embodiment.

FIG. 4A shows a delivery catheter and a lead according to an embodiment.

FIG. 4B shows a delivery catheter and a lead according to another embodiment.

FIG. 5A shows a delivery sheath and a lead prior to engagement of the sheath and lead according to an embodiment.

FIG. 5B shows a delivery sheath and a lead when the sheath and lead are engaged according to an embodiment.

FIG. 6 shows a flowchart of a method for introducing a lead into a subject.

### DETAILED DESCRIPTION

This disclosure is directed to leads for cardiac pacing, especially conduction system pacing, and delivery systems for such leads.

As defined herein, the phrase “distal” may refer to being situated away from a point of attachment (e.g., to a device such as the implantable pulse generator) or from an operator (e.g., a physician, a user, etc.). A distal end of a lead or a catheter may refer to an end of the lead or the catheter that is away from the operator or from a point of attachment to the implantable pulse generator.

As defined herein, the phrase “proximal” may refer to being situated nearer to a point of attachment (e.g., to a device such as the implantable pulse generator) or to an operator (e.g., a physician, a user, etc.). A proximal end of a lead or a catheter may refer to an end of the lead or the catheter that is close to the operator or to a point of attachment to the implantable pulse generator.

As defined herein, the phrase “French” may refer to a unit to measure the size (e.g., diameter or the like) of device such as a catheter, a lead, etc. For example, a round catheter or lead of one (1) French has an external diameter of ⅓ millimeters. For example, if the French size is 9, the diameter is 9/3=3.0 millimeters.

As defined herein, the phrase “helix” may refer to (e.g., an object) having a three-dimensional shape like that of a wire wound (e.g., in a single layer) around a cylinder or cone, as in a corkscrew or spiral staircase. The phrase “linear” may refer to being arranged in or extending straightly or nearly straightly.

As defined herein, the phrase “conductive” may refer to electrically conductive.

As defined herein, the phrase “septum” may refer to a partition separating two chambers, such as that between the chambers of the heart. Septum can be atrial septum and/or ventricular septum. The phrase “ventricular septum” or “inter-ventricular septum” may refer to a partition separating two ventricular chambers. The phrase “right ventricular septum” may refer to the ventricular septum where the RBB is located, while “left ventricular septum” may refer to the ventricular septum where the LBB is located.

As defined herein, the phrase “pacing” may refer to depolarization of the atria or ventricles, resulting from an impulse delivered (e.g., at desired voltage(s) for a desired duration, or the like) from a device (such as a pulse generator) down a lead to the heart via myocardium or directly via the cardiac conduction system. The phrase “sensing” may refer to detection by the device of intrinsic atrial or ventricular or conduction system electrical signals that are conducted up a lead. It will be appreciated that each of the electrode described herein can be configured as a pacing electrode and/or a sensing electrode. It will also be appreciated that each of the electrode described herein can be configured as anode and/or cathode.

As defined herein, the phrase “conduction system pacing” or “CSP” may refer to a therapy that involves the placement of permanent pacing leads along different sites or pathways of the cardiac conduction system with the intent of overcoming sites of atrioventricular conduction disease and delay, thereby providing a pacing solution that results in more synchronized biventricular activation. Lead placement for CSP can be targeted at the bundle of His, known as His-bundle pacing (HBP), at the region of the left bundle branch (LBB), known as LBB pacing (LBBP), or at the region of the right bundle branch (RBB), known as RBB pacing (RBBP) or both at the regions of RBB and LBB for Bi-lateral Bundle Branch Pacing (BBBP). Compared with conventional right ventricular (RV) pacing or biventricular (RV and left ventricular (LV)) pacing, where RV apical pacing lead and/or LV epicardial lead are implanted, the lead for CSP is placed through the septum e.g., closer to the His-bundle, the LBB, and/or the RBB. As such, the design, function, and purpose of the lead(s) for cardiac conduction system are different from those of the lead(s) for RV and/or LV pacing. It will be appreciated that ventricular pacing (e.g., RV pacing or the like) may be un-physiological and may result in adverse outcomes of mitral and/or tricuspid regurgitations, atrial fibrillation, heart failure, and/or pacing induced cardiomyopathy. CSP can be physiological pacing that can results in electrical-mechanical synchronization to mitigate chronic clinical detrimental consequence including e.g., pacing induced cardiomyopathy. It will also be appreciated that CSP indications may include e.g., a high burden of ventricular pacing being necessary (e.g., permanent atrial fibrillation with atrioventricular block, slowly conducted atrial fibrillation, pacing induced cardiomyopathy, atrioventricular node ablation, etc.); sick sinus syndrome, when atrioventricular node conduction diseases exist; and/or an alternative to biventricular pacing in heart failure patients with bundle branch block, narrow QRS and PR prolongation, biventricular pacing no-responders or patients need biventricular pacing cardiac resynchronization therapy upgrade, or the like.

Some embodiments of the present application are described in detail with reference to the accompanying drawings so that the advantages and features of the present application can be more readily understood by those skilled in the art. The terms “near”, “far”, “top”, “bottom”, “left”, “right”, and the like described in the present application are defined according to the typical observation angle of a person skilled in the art and for the convenience of the description. These terms are not limited to specific directions.

Processes described herein may include one or more operations, actions, or functions depicted by one or more blocks. It will also be appreciated that although illustrated as discrete blocks, the operations, actions, or functions described as being in various blocks may be divided into additional blocks, combined into fewer blocks, or eliminated, depending on the desired implementation. Any features described in one embodiment may be combined with or incorporated/used into the other embodiment, and vice versa. The scope of the disclosure should be determined by the appended claims and their legal equivalents, rather than by the examples given herein. For example, the steps recited in any method claims may be executed in any order and are not limited to the order presented in the claims. Moreover, no element is essential to the practice of the disclosure unless specifically described herein as “critical” or “essential.”

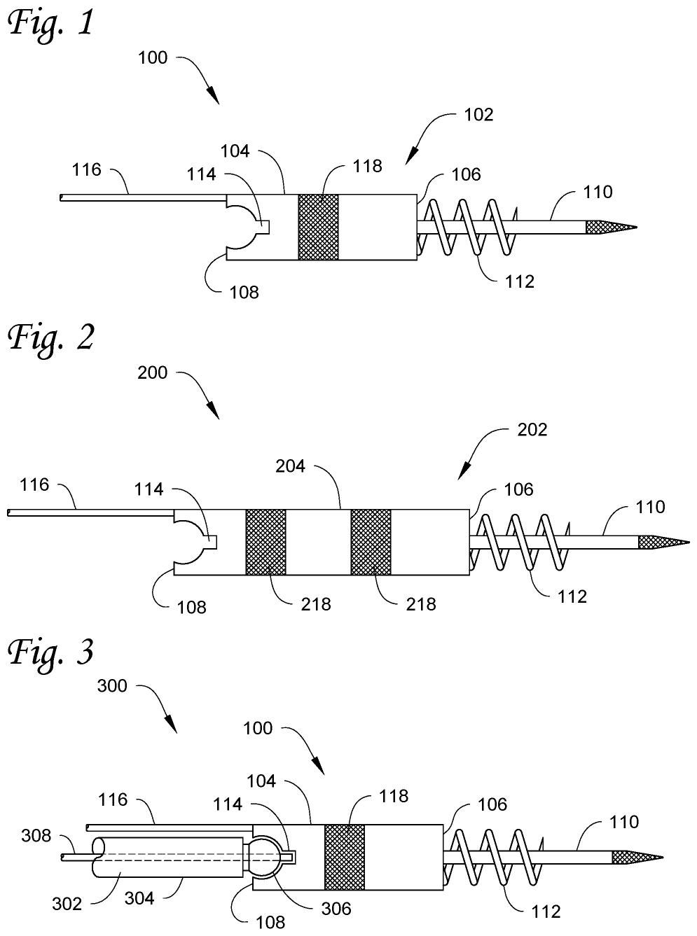

FIG. 1 shows a lead according an embodiment. Lead 100 includes lead distal tip 102 having lead distal tip body 104. Lead distal tip body 104 includes first end 106 and second end 108. A linear member 110 (e.g., a needle, a rod with tapered tip, or the like) and a helical member 112 extend from first end 106. Second end 108 includes engagement feature 114. Lead body 116 (e.g., a conductor, an insulated wire(s), or the like) extends from second end 108 to a proximal end (not shown) of the lead 100. A ring electrode 118 can be provided on an outer surface of lead distal tip body 104. In an embodiment, the ring electrode(s) can be optional.

Lead 100 is a lead used for providing electrical impulses to an organ or system and/or sensing electrical signals from the organ or system. In an embodiment, lead 100 is a CSP lead. In an embodiment, lead 100 is configured to provide electrical impulses to a subject at the bundle of His, known as His-bundle pacing (HBP), at the region of the left bundle branch (LBB), known as LBB pacing (LBBP), or at the region of the right bundle branch (RBB), known as RBB pacing (RBBP) or both at the regions of RBB and LBB for Bi-lateral Bundle Branch Pacing (BBBP). Compared with conventional right ventricular (RV) pacing or biventricular (RV and left ventricular (LV)) pacing, where RV apical pacing lead and/or LV epicardial lead via coronary sinus vein are implanted, the lead for CSP is placed through the septum e.g., closer to the His-bundle, the LBB, and/or the RBB.

Lead distal tip 102 is provided at a distal end of the lead 100. Lead distal tip 102 includes lead distal tip body 104 configured to support electrodes such as linear member 110, helical member 112, and/or ring lead body 118 in position such that the electrodes can provide electrical impulses. Lead distal tip body 104 can be, for example, a cylindrical body or any other suitable shape for lead distal tip 102 such that lead distal tip 102 can be securely and safely located within the subject. Lead distal tip body 104 includes a first end 106 at a distal end of the lead distal tip body 104 and a second end 108 that is proximal to a pulse generator used with lead 100. Lead distal tip 102 can be sized to reduce the impact of cardiac cycles on the position and/or security of attachment of the lead distal tip 102 to the target tissue or organ in the subject. The lead distal tip body 104 can be formed of flexible material such that the impact of cardiac cycles on the position and/or security or attachment of the lead distal tip 102 to the target tissue or organ in the subject can be reduced. Lead distal tip body 104 can be sized to have an outer diameter of 3 mm or less, or more preferable 2 mm or less. Lead distal tip body 104 can have a length of from approximately 5 mm to approximately 16 mm. In an embodiment, lead distal tip body 104 can include a drug-eluting coating. The drug-eluting coating can be any suitable coating capable of releasing a suitable drug over time, with such drugs including, as non-limiting examples, steroids or other anti-inflammatory drug compositions.

In embodiments, a lead distal tip such as the lead distal tip 102 can include any or all suitable features of a lead distal tip as described in U.S. patent application Ser. No. 17/804,705, which is herein incorporated by reference in its entirety.

A linear member 110 can be provided at the first end 106 of lead distal tip body 104. In an embodiment, linear member 110 is fixed with respect to lead distal tip body 104. In an embodiment, linear member 110 is extendable from lead distal tip body 104. In an embodiment, linear member 110 is retractable into lead distal tip body 104. In an embodiment, the linear member 110 is an electrode. The linear member 110 can be configured to provide electrical impulses, to sense electrical signals, or the like. In an embodiment, the linear member 110 is configured to provide alignment and/or support the attachment of lead distal tip 102 to the organ or system to which lead distal tip 102 is attached. Linear member 110 can have a pointed end. Linear member 110 can be positioned at a center of the first end 106 of lead distal tip body 104. Linear member 110 can be any suitable material for penetrating tissue and, in embodiments, providing electrical impulses. In an embodiment, the length of an inextensible (fixed length) linear element can be of the range of 0.5 millimeters (mm) to the length of 6 mm. Linear member 110 can be formed of, for example, platinum-iridium or tantalum. Linear member 110 can include a fractal coating. In an embodiment, the fractal coating is titanium nitride or iridium oxide. While linear member 110 is shown in FIG. 1, it is understood that a core can be used at the first end 106 of lead distal tip body 104. The core can be any suitable core as described in U.S. patent application Ser. No. 17/804,705, which is herein incorporated by reference in its entirety. For example, the core can be a conical, frustoconical, linear, or any other suitable shape member disposed within a space defined by helical member 112.

Helical member 112 can be provided at first end 106 of lead distal tip body 104, in place of or in addition to linear member 110. In an embodiment, helical member 112 is fixed with respect to lead distal tip body 104. In an embodiment, helical member 112 is extendable from lead distal tip body 104. In an embodiment, helical member 112 is retractable into lead distal tip body 104. In an embodiment, helical member 112 is an electrode. The helical member 112 can be configured to provide electrical impulses, to sense electrical signals, or the like. In an embodiment, helical member 112 is configured to be threaded into tissue to secure the position of lead distal tip 102. In an embodiment, the helical member 112 is pre-torqued such that the helical member 112 can be released at the target location such that the helical member 112 can attach at the target location. In an embodiment, helical member 112 can be a shape-memory alloy configured to use the shape-memory properties to adopt a suitable shape for attachment at the target location. In embodiments including both helical member 112 and linear member 110, the helical member 112 can radially surround the linear member 110. In an embodiment, an inner diameter of the helical member 112 can be greater than an outer diameter of the linear member 110. Helical member 112 can be any suitable material for penetrating tissue and, in embodiments, providing electrical impulses. In an embodiment, a stopper can be provided in linear member 110, helical member 112, and/or at first end 106 configured to reduce or prevent perforation of the target location by the linear member 110 and/or helical member 112. Helical member 112 can be formed of, for example, platinum-iridium or tantalum. Helical member 112 can include a fractal coating. In an embodiment, the fractal coating is titanium nitride or iridium oxide.

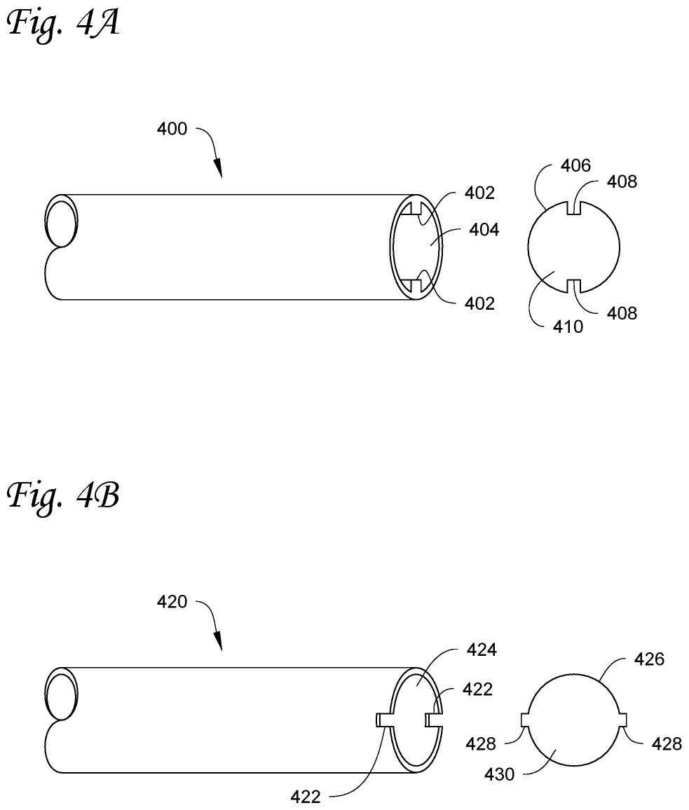

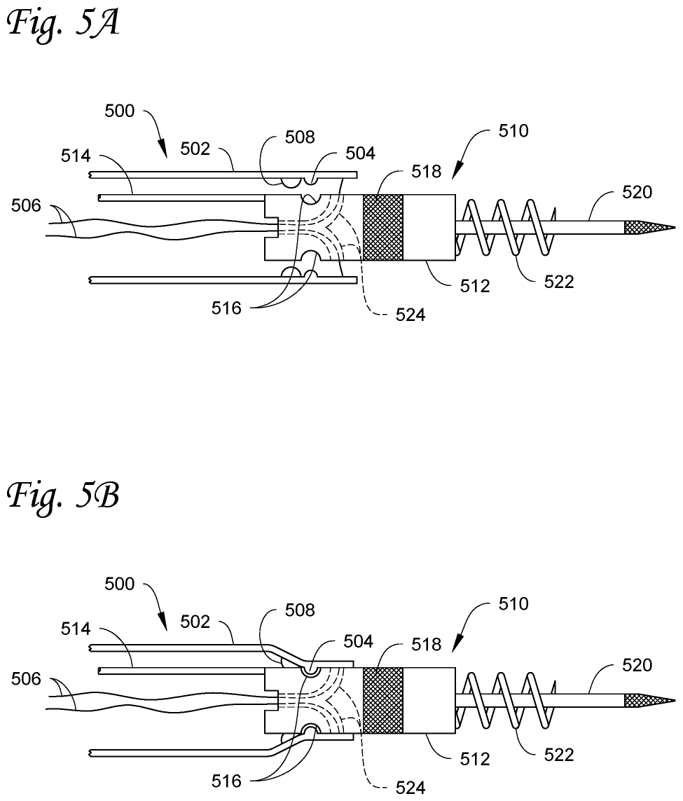

Lead distal tip body 104 includes engagement feature 114. The engagement feature 114 is a feature configured to engage with a corresponding feature on a delivery tool such as, as non-limiting examples, a manual surgical tool, a robot, a delivery catheter, a delivery sheath, or the like. The engagement feature 114 is configured such that the lead distal tip 102 can be manipulated by the delivery tool. Manipulation of the lead distal tip 102 by way of the engagement feature 114 can include translational movement and/or rotation of lead distal tip 102 and, optionally, extension or retraction of the linear member 110 and/or helical member 112. In the embodiment shown in FIG. 1, the engagement feature 114 is a socket formed in second end 108, the socket configured to receive a projection provided on the delivery tool. In the embodiment shown in FIG. 1, the engagement 114 further includes an interface configured to receive a stylet, such that the stylet can be used to manipulate the linear member 110 and/or the helical member 112 such that the linear member 110 and/or the helical member 112 can be extended or retracted through use of the stylet. In embodiments, the engagement feature 114 provided on lead distal tip body 104 can include one or more projections at second end 108 as shown in FIG. 4B and described below, recesses at second end 108 as shown in FIG. 4A and described below. In an embodiment, the engagement feature 114 can include features on an outer surface of the lead distal tip body 104, such as recesses as shown in FIGS. 5A and 5B and described below.

Lead body 116 is a lead body configured to convey electrical signals including sensed electrical signals and/or impulses generated at a device such as a pulse generator, for example an implantable pulse generator of a pacemaker, to the lead distal tip 102. Lead body 116 can connect to the lead distal tip 102 at a second end 108 of the lead distal tip body 104. In an embodiment, lead body 116 can connect to the lead distal tip 102 at or near a perimeter of the second end 108. In an embodiment, the lead body is positioned such that it does not interfere with engagement feature 114 or the engagement thereof with a delivery tool. Lead body 116 includes conductive line(s) and an insulating coating. Each conductive line extends to a proximal end (not shown) of the lead 100, and can be configured to connect the corresponding linear member, the corresponding helical member, or the corresponding ring electrode (e.g., 218 of FIG. 2) to a connector (e.g., an IS-1 connector, an IS-4 connector, or a DF-4 connector, or the like) at the proximal end of the lead 100. The insulating coating can be selected for biocompatibility, for example to reduce or prevent inflammation, encapsulation, or other bodily responses to the presence of the lead body 116 extending through the body of the subject to the lead distal tip 102. The lead body 116, for example can be an insulated wire. The lead body 116 is selected for a high degree of flexibility, to reduce the impact of cardiac cycles on the position of lead distal tip 102. In an embodiment, the lead body 116 is a thin wire, for example a wire having an outer diameter of 2 French or less. In an embodiment, the lead body 116 has an outer diameter of 1.5 French or less.

Ring electrode 118 can be provided on an outer surface of lead distal tip body 104, between first end 106 and second end 108. Ring electrode 118 is conductive material formed connected to a lead body such that ring electrode 118 can provide electrical impulses and/or can sense electrical signals where the lead distal tip body 104 is attached. Ring electrode 118 can be any suitable conductive material. Ring electrode can be formed of any suitable conductive material, with non-limiting examples including alloy materials such as MP35N alloy or an alloy of MP35N further including silver, for example including silver in an amount between 25% and 41% by mass.

FIG. 2 shows a lead according to another embodiment. In the embodiment shown in FIG. 2, lead 200 includes a lead distal tip 202 including a plurality of the ring electrodes 218 provided on the outer surface of lead distal tip body 204. Each of the plurality of ring electrodes 218 can be provided on the outer surface of the lead distal tip body 204 such that the ring electrodes 218 are spaced apart along an axial direction of the lead distal tip body 204, with at least a portion of the lead distal tip body 204 being provided between each of the plurality of ring electrodes 218. The other features of lead distal tip 202 including first end 106, second end 108, linear member 110, helical member 112, engagement feature 114, and lead body 116 can be the same as those features shown in FIG. 1 and described above.

In an embodiment, the linear member and/or the helical member can be a single polar electrode or a bipolar electrode. In an embodiment, the ring electrode(s) can be positioned outside the septum. In another embodiment, the ring electrode(s) can be positioned inside the septum at or near the RBB. In yet another embodiment, one ring electrode can be positioned outside the septum and another ring electrode can be positioned inside the septum at or near the RBB. In yet another embodiment, the ring electrode(s) can be optional. In an embodiment, the helix element can be positioned inside the septum at or near the RBB and the linear member can be positioned inside the septum at or near the LBB. In another embodiment, the helix element and the linear member can be positioned inside the septum at or near the LBB (and a ring electrode can be positioned inside the septum at or near the RBB). Other embodiments (including the structure, material, dimension, configuration, arrangement, and/or operations) of the lead distal tip (such as the linear member, the helical member, and/or the stopper, or the like) and/or the delivery catheter can be found in the U.S. patent application Ser. No. 17/804,705, which is incorporated herein by reference in its entirety.

FIG. 3 shows a lead and a delivery system according to an embodiment. Lead and delivery system 300 includes lead 100 as described above and shown in FIG. 1, engaged with a delivery catheter 302. The delivery catheter 302 includes catheter body 304, engagement feature 306, and stylet 308.

Lead and delivery system 300 is configured to direct lead 100 to a target location in the subject and attach the lead 100 at that target location. Delivery catheter 302 is configured to connect to lead 100 such that lead 100 can be steered through the body of the subject, for example to arrive at suitable target within the subject such as the interventricular septum (IVS) or the interatrial septum (IAS) of the subject. The delivery catheter includes catheter body 304. Catheter body 304 has sufficient length, thickness, and rigidity and flexibility properties to be inserted into the subject and direct lead 100 to the target location. Engagement feature 306 is disposed at a distal end of the catheter body 304. In an embodiment, catheter body 304 can be configured to apply rotational torque to the lead 100, for example using a torque shaft included in or extending through catheter body 304. In the embodiment shown in FIG. 3, the engagement feature 306 is a projection configured to engage with a socket provided as the corresponding engagement feature 114 provided on lead 100. The engagement feature 306 can be any suitable engagement feature selected based on the ability to engage the corresponding engagement feature 114 of the lead 100, for example according to the arrangements shown in FIGS. 4A-5B and described below. The engagement feature 306 can alternatively be, for example, projections, notches, recesses, a combination of projections disposed on a deflectable end of the delivery catheter along with grasping lines, an inflatable segment configured to interface with engagement feature 114 when inflated, a cup capable of receiving a portion of lead 100, or the like.

Stylet 308 can be included in engagement feature 306. Stylet 308 is configured to engage with lead 100 such that components of lead 100 can be actuated, such as linear member 110 and/or helical member 112. Stylet 308 is configured such that the stylet 308 can be manipulated to actuate the components of lead 100 by a user of the delivery catheter 302, for example by connection to an actuator at the proximal end (not shown) of the delivery catheter 302 controlled by the user. In embodiments, the stylet 308 can be configured to trigger shape changes in shape memory materials used in lead 100, for example shape memory materials in the linear member 110 and/or helical member 112.

FIG. 4A shows a delivery catheter and a lead according to an embodiment. In FIG. 4A, delivery catheter 400 includes inwards projections 402 at a distal end 404 of the delivery catheter 400. Lead distal tip 406 includes recesses 408 formed at a proximal end 410 of the lead distal tip 406. Inwards projections 402 extend from an inner surface of a wall of the delivery catheter 400 towards a center of an internal space within the delivery catheter 400. The inwards projections 402 provided at distal end 404 of the delivery catheter 400 are configured to be accommodated within and engage with the recesses 408 formed in the lead distal tip 406. The engagement of the projections 402 with the recesses 408 when the delivery catheter 400 and the lead distal tip 406 are joined to one another can allow the steering and rotation of the lead distal tip 406 through manipulation of the delivery catheter 400. The inwards projections 402 can be sized in height, length, cross-sectional shape, and the like such that the inwards projections have sufficient rigidity and sufficient contact with recesses 408 to be capable of steering and/or rotating the lead distal tip 406. The recesses 408 can be sized and shaped to engage with the inwards projections 402. The proximal end 410 of the lead distal tip 406 can be sized and shaped such that a portion of the lead distal tip 406 at or near the proximal end 410 can be accommodated within delivery catheter 400 at distal end 404. A stylet can be used to push against the lead distal tip while disengaging the delivery catheter from the distal lead.

FIG. 4B shows a delivery catheter and a lead according to another embodiment. In FIG. 4D, delivery catheter 420 includes notches or recesses 422 at a distal end 424 of the delivery catheter 420. Lead distal tip 426 includes projections 428 formed at a proximal end 430 of the lead distal tip 426. Projections 428 extend from an outer surface of the lead distal tip 426. The projections 428 provided at proximal end 430 of the lead distal tip 426 are configured to be accommodated within and engage with recesses or notches 422 formed in the delivery catheter 420. In an embodiment, the delivery catheter 420 includes notches 422 that are cut out of the end of the delivery catheter 420. In an embodiment, recesses 422 can be provided, the recesses 422 being formed in an inner surface of the delivery catheter 420. The engagement of the projections 428 with the notches or recesses 422 when the delivery catheter 420 and the lead distal tip 426 are joined to one another can allow the steering and rotation of the lead distal tip 426 through manipulation of the delivery catheter 420. The projections 428 can be sized in height, length, cross-sectional shape, and the like such that the inwards projections have sufficient rigidity and sufficient contact with notches or recesses 422 to be capable of steering and/or rotating the lead distal tip 426. The notches or recesses 422 can be sized and shaped to engage with the projections 428. The proximal end 430 of the lead distal tip 426 can be sized and shaped such that a portion of the lead distal tip 426 at or near the proximal end 430 can be accommodated within delivery catheter 420 at distal end 424.

FIG. 5A shows a delivery sheath and a lead prior to engagement of the sheath and lead according to an embodiment. Delivery sheath 500 includes sheath body 502, rigid projections 504, grasping lines 506, and optionally resilient members 508. The lead distal tip 510 includes lead distal tip body 512, lead body 514, recesses 516, ring electrode 518, linear member 520 and helical member 522. Optionally, lead distal tip 510 can include channels 524.

Delivery sheath 500 is configured to facilitate delivery of lead distal tip 510 to a target location within a subject. Delivery sheath 500 can be included in or attached to a delivery tool, such as being included in or provided at an end of a delivery catheter, included in or deployed by a robot, or the like. The delivery sheath includes sheath body 502. Sheath body 502 can be generally cylindrical in shape. Sheath body 502 is sized and shaped to define an internal space, for example being a hollow cylinder. The internal space is sized such that the lead distal tip can be readily accommodated within sheath body 502. In an embodiment, sheath body 502 has an internal diameter that is greater than an outer diameter of the lead distal tip body 512. Sheath body 502 extends to a distal end. The distal end of sheath body 502 is deflectable such that the distal end of the sheath body 502 can be drawn inwards such that projections 504 and optionally a portion of the inner surface of sheath body 502 and/or resilient members 508 can be brought into contact with distal tip body 512. In a state where the deflectable distal end of the sheath body 502 is not deflected, the lead distal tip 510 can be readily inserted into or removed from the delivery sheath 500. In an embodiment, sheath body 502 can include slits, notches, or other features at the deflectable distal end to facilitate the deflection of the sheath body 502 at said deflectable distal end.

Projections 504 are provided on the deflectable distal end of sheath body 502. The projections are sized and shaped such that they fit into recesses 516 provided on the lead distal tip 510. The projections 504 can be rigid and sized such that the projections 504 can fit stably and securely within recesses 516, such that engagement between projections 504 and recesses 516 can be used to steer, drive, rotate, and/or otherwise move the lead distal tip 510 through manipulation of the delivery sheath 500.

Grasping lines 506 can be joined to inner surfaces of the sheath body 502 at the deflectable distal end. The grasping lines 506 are configured to allow the deflectable distal end of sheath body 502 to be drawn inwards such that the projections 504 engage with recesses 516. The grasping lines 506 can be threads configured to be pulled with sufficient tension to cause suitable deflection of the distal end of the sheath body. The grasping lines can be any suitable material that is biocompatible and capable of forming threads capable of applying sufficient tension to draw the deflectable distal end inwards. In an embodiment, the grasping lines 506 can be threaded through one or more channels 524 formed in the lead distal tip 510.

Resilient members 508 can optionally be included on the inner surface of sheath body 502. The resilient members can include a compressible material such as an elastic material. When the deflectable ends of sheath body 502 are drawn towards lead distal tip 510, the resilient members can be compressed against lead distal tip body 512. Compression of the resilient members 508 against lead distal tip body 512 can assist in retaining and stabilizing lead distal tip 510 when it is engaged with delivery sheath 500. The resilient members 508 can have any suitable shape, for example, being a plurality of projections from the inner surface of sheath body 502, a ring disposed on the inner surface of sheath body 502, or the like. Resilient members 508 can be any suitable compressible material, such as biocompatible elastomeric materials or the like. The grasping element at the distal end of the delivery catheter can instead be made of memory alloy to achieve the same clamping/grasping function as described above.

Lead distal tip 510 includes lead distal tip body 512 configured to support electrodes such as linear member 520, helical member 522, and/or ring lead body 518 in position such that the electrodes can provide electrical impulses. Lead distal tip body 512 can be, for example, a cylindrical body or any other suitable shape for lead distal tip 510 such that lead distal tip 510 can be securely and safely located within the subject. Lead distal tip body 512 includes a distal end and a proximal end, defined with respect to a pulse generator used with lead distal tip 510. Lead distal tip 510 can be sized to reduce the impact of cardiac cycles on the position and/or security of attachment of the lead distal tip 510 to the target tissue or organ in the subject. The lead distal tip body 512 can be formed of flexible material such that the impact of cardiac cycles on the position and/or security or attachment of the lead distal tip 510 to the target tissue or organ in the subject.

Lead body 514 is a lead body configured to convey electrical impulses generated at a device such as a pulse generator, for example an implantable pulse generator of a pacemaker, to the lead distal tip 510. Lead body 514 can connect to the lead distal tip 510 at a proximal end of the lead distal tip body 512. In an embodiment, lead body 514 can connect to the lead distal tip 510 at or near a perimeter of the distal end thereof. Lead body 514 includes a conductive line and an insulating coating. The insulating coating can be selected for biocompatibility, for example to reduce or prevent inflammation, encapsulation, or other bodily responses to the presence of the lead body 514 extending through the body of the subject to the lead distal tip 510. The lead body 514, for example can be an insulated wire. The lead body 514 is selected for a high degree of flexibility, to reduce the impact of cardiac cycles on the position of lead distal tip 510. In an embodiment, the lead body 514 is a thin wire, for example a wire having an outer diameter of 2 French or less.

Recesses 516 can be formed into the outer surface of lead distal tip body 512. The recesses 516 can be sized, shaped, and distributed about the outer surface of lead distal tip body 512 such that projections 504 can be received in the recesses 516. The accommodation of projections 504 in recesses 516 can be a fit configured such that the lead distal tip can be manipulated, for example guided, rotated, moved, and the like by way of the engagement between projections 504 and recesses 516 and movement or rotation of the delivery sheath 500.

Ring electrode 518 can be provided on an outer surface of lead distal tip body 512. Ring electrode 518 is conductive material formed connected to a lead body such that ring electrode 518 can provide electrical impulses to the body where the lead distal tip body 510 is attached. Ring electrode 518 can be any suitable conductive material.

Linear member 520 can be provided at the distal end of lead distal tip body 512. In an embodiment, linear member 520 is fixed with respect to lead distal tip body 512. In an embodiment, linear member 520 is extendable from lead distal tip body 512. In an embodiment, linear member 520 is retractable into lead distal tip body 512. In an embodiment, the linear member 520 is an electrode configured to provide electrical impulses. In an embodiment, the linear member 520 is configured to provide alignment and/or support the attachment of lead distal tip 510 to the organ or system to which lead distal tip 510 is attached. Linear member 520 can have a pointed end. Linear member 520 can be positioned at a center of the distal end of lead distal tip body 512. Linear member 520 can be any suitable material for penetrating tissue and, in embodiments, providing electrical impulses. In the embodiment shown in FIGS. 5A and 5B, the linear member 520 has a ball tip. In an embodiment, the ball tip has a diameter greater than the diameter of the preceding portion of linear member 520. In an embodiment, the ball tip has a diameter equal to or less than that of the preceding portion of linear member 520, so as to present a hemispherical or blunted end to the linear member 520. It is understood that the ball tip as shown in FIGS. 5A and 5B can be used in any linear member as described herein, such as the linear member 110 described above and shown in FIGS. 1-3.

Helical member 522 can be provided at distal end of lead distal tip body 512, in place of or in addition to linear member 520. In an embodiment, helical member 522 is fixed with respect to lead distal tip body 512. In an embodiment, helical member 522 is extendable from lead distal tip body 512. In an embodiment, helical member 522 is retractable into lead distal tip body 512. In an embodiment, helical member 522 is an electrode configured to provide electrical impulses. In an embodiment, helical member 522 is configured to be threaded into tissue to secure the position of lead distal tip 510. In an embodiment, the helical member 522 is pre-torqued such that the helical member 522 can be released at the target location such that the helical member 522 can attach at the target location. In an embodiment, helical member 522 can be a shape-memory alloy configured to use the shape-memory properties to adopt a suitable shape for attachment at the target location. In embodiments including both helical member 522 and linear member 520, the helical member 522 can radially surround the linear member 520. Helical member 522 can be any suitable material for penetrating tissue and, in embodiments, providing electrical impulses. In an embodiment, a stopper can be provided in linear member 520, helical member 522, and/or at distal end configured to reduce or prevent perforation of the target location by the linear member 520 and/or helical member 522.

Channels 524 can be formed in the lead distal tip body 512. In an embodiment, the channel(s) 254 can extend from the recess(es) 516 (see FIG. 5B) to the socket (e.g., 114 of FIG. 1). Channels 524 can be configured to allow the grasping lines 506 to be threaded through the channels 524 such that the grasping lines 506 can pull the deflectable ends of the delivery sheath 500 towards the outer surface of lead distal tip body 512 such that the projections 504 contact the lead distal tip body 512. For example, channels 524 can each include a first opening on an outer surface of the lead distal tip body 512, a second opening at the proximal end of lead distal tip body 512, and extend through the lead distal tip body 512 from the first opening to the second opening.

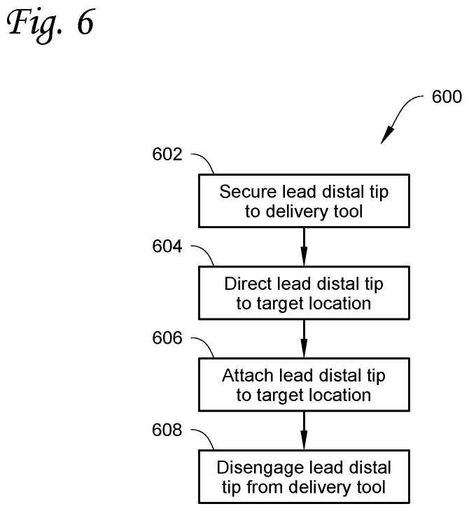

FIG. 5B shows a delivery sheath and a lead when the sheath and lead are engaged according to an embodiment. In the embodiment shown in FIG. 5B, grasping lines 506 are under tension such that the ends of the sheath body 502 are pulled inwards such that rigid projections 504 interface with recesses 516 provided on the lead distal tip 510. The optional resilient members 508 can be compressed against the outer surface of lead distal tip body 512. Engagement of the rigid projections 504 with recesses 516 and the optional compression of resilient members 508 can secure lead distal tip 510 to the delivery sheath 500, such that manipulation of the delivery sheath 500 can be used to direct lead distal tip 510. Once the lead distal tip 510 is in place, grasping lines 506 can be released such that the ends of sheath body 502 return to the position in FIG. 5A and the FIG. 6 shows a flowchart of a method for introducing a lead into a subject. Method 600 includes securing a lead distal tip to a delivery tool 602, using the delivery tool to direct the lead distal tip to a target location 604, attaching the lead distal tip to heart tissue at the target location 606, and disengaging the lead distal tip from the delivery tool 608.

The lead distal tip is secured to a delivery tool at 602. The lead distal tip can be any lead distal tip described herein, such as lead distal tip 100 described above and shown in FIG. 1. The lead distal tip includes a lead body extending from the lead distal tip. The delivery tool can be any suitable tool for manipulating and directing the lead distal tip, such as a robotic tool or any delivery catheter or delivery sheath described herein. The securement of the lead distal tip to the delivery tool can be through any suitable engagement of engagement features respectively provided on the lead distal tip and the delivery tool, for example including engagement of corresponding projections and recesses, engagement at socket interfaces, use of grasping lines, or the like.

The lead distal tip is directed to a target location at 604. The lead distal tip can be guided through manipulation of the delivery tool, such as pushing and steering of a delivery catheter, movement of a robot delivery tool, or the like. The target location can be any suitable location for attachment of the lead distal tip. The target location can be selected based on the use of the lead distal tip, for example as lead tips for conduction system pacing (CSP). In an embodiment, the target location is the interventricular septum (IVS) or the interatrial septum (IAS) of the subject.

The lead distal tip can be attached to heart tissue of the subject at the target location at 606. The attachment can be, for example, provided by extending a linear member and/or a helical member of the lead distal tip, such as linear member 110 and/or helical member 112 of the lead distal tip 100 described above and shown in FIG. 1. In an embodiment, extension, retraction, and/or rotation of the linear member, helical member, or any other such suitable feature of the lead distal tip can be driven using a stylet provided in the delivery tool and interface of the stylet with features included in the lead distal tip.

When the lead distal tip is attached to the subject at the target location, the delivery tool and the lead distal tip can be disengaged from one another at 608. The disengagement can be through any suitable method for the respective engagement features included in the lead distal tip and the delivery too. For example, grasping lines can be released, the delivery tool can be retracted and/or rotated, socket features can be adjusted to release the lead distal tip, combinations thereof, or the like can be performed to disengage the lead distal tip from the delivery tool. The delivery tool can be removed from the subject while the lead distal tip remains attached at the target location. Following the disengagement of the lead distal tip from the delivery tool, the lead distal tip can be used to provide pacing to the subject, such as CSP pacing.

Aspects:

It is understood that any of aspects 1-9 can be combined with any of aspects 10-16 or 17-19. It is understood that any of aspects 10-16 can be combined with any of aspects 17-19.

Aspect 1. A lead distal tip for a pacemaker, comprising:
a lead distal tip body having a first end and a second end, opposite the first end;
a linear member disposed at the first end;
a helical member surrounding the linear member;
a ring electrode disposed on the lead distal tip body; and
a lead body extending from the second end, wherein the lead body has an outer diameter of 2 French or less.

Aspect 2. The lead distal tip according to aspect 1, further comprising an engagement feature configured to engage with a delivery tool.

Aspect 3. The lead distal tip according to aspect 2, wherein the engagement feature includes a socket formed at the second end.

Aspect 4. The lead distal tip according to aspect 3, wherein the socket is configured to receive a stylet of the delivery tool.

Aspect 5. The lead distal tip according to any of aspects 2-4, wherein the engagement feature includes a plurality of depressions in an outer surface of the lead distal tip body.

Aspect 6. The lead distal tip according to any of aspects 2-5, wherein the engagement feature includes a plurality of projections disposed on an outer surface of the lead distal tip body.

Aspect 7. The lead distal tip according to any of aspects 1-6, wherein the linear member is extendable and retractable.

Aspect 8. The lead distal tip according to any of aspects 1-7, wherein the helical member is extendable and retractable.

Aspect 9. The lead distal tip according to any of aspects 1-8, further comprising a second ring electrode disposed on the lead distal tip body.

Aspect 10. A pacemaker lead and delivery system, comprising:
a lead distal tip including:
   a lead distal tip body having a first end and a second end, opposite the first end;
   a linear member disposed at the first end;
   a helical member surrounding the linear member;
   a ring electrode disposed on the lead distal tip body;
   a lead body extending from the second end, wherein the lead body has an outer diameter of 2 French or less; and
   a tip engagement feature; and
a lead delivery catheter including:
   a rigid body segment; and
   a delivery catheter engagement feature configured to engage the tip engagement feature.

Aspect 11. The pacemaker lead and delivery system according to aspect 10, wherein the tip engagement feature includes a socket and the delivery catheter engagement feature includes a stylet.

Aspect 12. The pacemaker lead and delivery system according to aspect 11, wherein at least one of the linear member and the helical member are extendable and retractable, and wherein the stylet is configured to extend or retract said at least one of the linear member and the helical member.

Aspect 13. The pacemaker lead and delivery system according to any of aspects 10-12, wherein the delivery catheter engagement feature includes recesses in an inner surface of a distal end of the lead delivery catheter, and the tip engagement feature includes projections from an outer surface of the lead distal tip body.

Aspect 14. The pacemaker lead and delivery system according to any of aspects 10-13, wherein the delivery catheter engagement feature includes projections from an inner surface of the lead delivery catheter, and the tip engagement feature includes recesses formed in an outer surface of the lead distal tip body.

Aspect 15. The pacemaker lead and delivery system according to aspect 14, wherein a distal end of the delivery catheter is configured to be deflected such that the delivery catheter engagement features interface with the tip engagement features.

Aspect 16. The pacemaker lead and delivery system according to aspect 15, further comprising grasping lines extending from the distal end of the delivery catheter, the grasping lines configured to deflect the distal end of the delivery catheter when tension is applied to the grasping lines.

Aspect 17. A method of implanting a pacemaker lead in a subject, comprising:
engaging tip engagement features of a lead distal tip with delivery catheter engagement features of a delivery catheter;
directing the lead distal tip to a target location while the tip engagement features and the delivery catheter engagement features are engaged;
attaching the lead distal tip to heart tissue of the subject at the target location; and
disengaging the tip engagement features from the delivery catheter engagement features after the lead distal tip is attached to the heart tissue,
wherein the lead distal tip further includes:
   a lead distal tip body having a first end and a second end, opposite the first end;
   a linear member disposed at the first end;
   a helical member surrounding the linear member;
   a ring electrode disposed on the lead distal tip body;
   a lead body extending from the second end, wherein the lead body has an outer diameter of 2 French or less.

Aspect 18. The method according to aspect 17, wherein the target location is interventricular septum (IVS) or interatrial septum (IAS) of the subject.

Aspect 19. The method according to aspect 17 or aspect 18, wherein attaching the lead distal tip to the heart tissue of the subject includes extending one or more of the linear member and the helical member into the heart tissue.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A lead distal tip for a pacemaker lead, comprising:
a lead distal tip body having a first end and a second end, opposite the first end;
a linear member disposed at the first end;
a helical member surrounding the linear member;
a ring electrode disposed on the lead distal tip body; and
a lead body extending from the second end, wherein the lead body has an outer diameter of 2 French or less.

2. The lead distal tip of claim 1, further comprising an engagement feature configured to engage with a delivery tool.

3. The lead distal tip of claim 2, wherein the engagement feature includes a socket formed at the second end.

4. The lead distal tip of claim 3, wherein the socket is configured to receive a stylet of the delivery tool.

5. The lead distal tip of claim 2, wherein the engagement feature includes a plurality of depressions in an outer surface of the lead distal tip body.

6. The lead distal tip of claim 2, wherein the engagement feature includes a plurality of projections disposed on an outer surface of the lead distal tip body.

7. The lead distal tip of claim 1, wherein the linear member is extendable and retractable.

8. The lead distal tip of claim 1, wherein the helical member is extendable and retractable.

9. The lead distal tip of claim 1, wherein the helical member is fixed.

10. A pacemaker lead and delivery system, comprising:
a lead distal tip including:
a lead distal tip body having a first end and a second end, opposite the first end;
a linear member disposed at the first end;
a helical member surrounding the linear member;
a ring electrode disposed on the lead distal tip body;
a lead body extending from the second end, wherein the lead body has an outer diameter of 2 French or less; and
a tip engagement feature; and
a lead delivery catheter including:
a rigid body segment; and
a delivery catheter engagement feature configured to engage the tip engagement feature.

11. The pacemaker lead and delivery system of claim 10, wherein the tip engagement feature includes a socket and the delivery catheter engagement feature includes a stylet.

12. The pacemaker lead and delivery system of claim 11, wherein at least one of the linear member and the helical member are extendable and retractable, and wherein the stylet is configured to extend or retract said at least one of the linear member and the helical member.

13. The pacemaker lead and delivery system of claim 10, wherein the delivery catheter engagement feature includes recesses in an inner surface of a distal end of the lead delivery catheter, and the tip engagement feature includes projections from an outer surface of the lead distal tip body.

14. The pacemaker lead and delivery system of claim 10, wherein the delivery catheter engagement feature includes projections from an inner surface of the lead delivery catheter, and the tip engagement feature includes recesses formed in an outer surface of the lead distal tip body.

15. The pacemaker lead and delivery system of claim 14, wherein a distal end of the delivery catheter is configured to be deflected such that the delivery catheter engagement feature interfaces with the tip engagement feature.

16. The pacemaker lead and delivery system of claim 15, further comprising grasping lines extending from the distal end of the delivery catheter, the grasping lines configured to deflect the distal end of the delivery catheter when tension is applied to the grasping lines.

17. A method of implanting a pacemaker lead in a subject, comprising:
engaging tip engagement features of a lead distal tip with delivery catheter engagement features of a delivery catheter;
directing the lead distal tip to a target location while the tip engagement features and the delivery catheter engagement features are engaged;
attaching the lead distal tip to heart tissue of the subject at the target location; and
disengaging the tip engagement features from the delivery catheter engagement features after the lead distal tip is attached to the heart tissue,
wherein the lead distal tip further includes:
a lead distal tip body having a first end and a second end, opposite the first end;
a linear member disposed at the first end;
a helical member surrounding the linear member;
a ring electrode disposed on the lead distal tip body;
a lead body extending from the second end, wherein the lead body has an outer diameter of 2 French or less.

18. The method of claim 17, wherein the target location is interventricular septum (IVS) or interatrial septum (IAS) of the subject.

19. The method of claim 17, wherein attaching the lead distal tip to the heart tissue of the subject includes extending one or more of the linear member and the helical member into the heart tissue.

20. A lead distal tip for a pacemaker lead, comprising:
a lead distal tip body having a first end and a second end, opposite the first end;
a core disposed at the first end;
a helical member surrounding the core; and
a lead body extending from the second end, wherein the lead body has an outer diameter of 2 French or less.
